(19) United States
(12) Patent Application Publication
(10) Pub. No.: US 2014/0128990 A1
(43) Pub. Date: May 8, 2014

(54) BONE PUTTY

(71) Applicants: David Franklin Farrar, York (GB); Nicola Jayne MacAuley, York (GB); John Rose, Collierville, TN (US)

(72) Inventors: David Franklin Farrar, York (GB); Nicola Jayne MacAuley, York (GB); John Rose, Collierville, TN (US)

(73) Assignee: SMITH & NEPHEW, INC., Memphis, TN (US)

(21) Appl. No.: 14/110,087

(22) PCT Filed: Apr. 4, 2012
(86) PCT No.: PCT/US12/32066

§ 371 Date: Jan. 16, 2014

(30) Foreign Application Priority Data
Apr. 4, 2011 (GB) 105621.5
Apr. 4, 2011 (GB) 105642.1

(51) Int. Cl. A61L 24/00; A61L 24/04; A61F 2/28

(52) U.S. Cl. CPC A61L 24/0036; A61F 2/28; A61L 24/046; A61L 24/0042; A61L 24/0084
USPC 623/23.58

(57) ABSTRACT

The present invention relates to a macroporous material for filling bone voids. In particular, we describe an implant material comprising bioresorbable polymer granules and a biocompatible water-miscible solvent, wherein the solvent at least partially dissolves and/or softens the polymer granules to form a mouldable mass that can be used to fill a bone defect but hardens when water is added and/or the implant material is placed in an aqueous environment, and wherein the implant material has macroporosity suitable for bone in-growth.

The present invention relates to bone void fillers. In particular, the present invention relates to a macroporous material for filling bone voids.

The present invention concerns macroporous materials for bone repair and bone void filling. Ideally, the material should be mouldable/formable so that it can fill and conform to irregular shaped and sized bone defects. However, once implanted it ideally should set hard so that the implant material maintains its shape and, under some circumstances, be able to bear loads. The material should not break up and needs to be tough. Furthermore, the material should allow rapid bone in-growth and, ultimately, be degradable and fully replaced by bone. In order to facilitate bone repair the material may incorporate a drug or bioactive molecule which is released to stimulate bone healing and repair.

A number of bone void filler materials are known, but very few meet all the ideal requirements.

Poly(methyl methacrylate) bone cements are widely used to fixate joint replacements but these materials are non-porous and non-degradable so they are not replaced by bone. In addition, when the cement cures, heat is generated and the temperature of the material can rise to 90° C. or above. This can damage any drug material or bioactive agent which have been added to the cement, particularly if the bioactive agent consist of proteins such as bone morphogeneic protein (BMP) etc.

Calcium phosphate ceramics, such as hydroxyapatite and tricalcium phosphate, are widely used for bone void filling. These fillers are available in a number of forms. For example, the use of dense and porous granules is known. These can be used to fill irregular shaped defects and allow bone growth into and between the granules. However, they cannot maintain a specific shape or form, and tend to migrate if not fully contained. Porous blocks in pre-formed shapes are also known. However, whilst these kinds of fillers maintain their shape, they cannot be used to fill irregular sized/shaped defects.

In addition, it is not easy to incorporate a drug or bioactive material into these ceramics as high temperatures are required in their manufacture. Drugs or bioactive agents can be adsorbed or coated onto the surface of these ceramics but they tend to be released very quickly.

Calcium phosphate cements have also been used as bone fillers. These kinds of fillers have the advantage of being mouldable, and even injectable, and once in place they set hard. However, whilst they may contain micropores, these tend not to allow significant levels of bone ingrowth. Some calcium phosphate cements have macropores but these generally compromise the mechanical strength of the material. In addition, calcium phosphate ceramics (blocks, cements etc) generally tend to form brittle materials.

There have been attempts to produce bone void fillers which harden in-situ; these combine ceramic granules with a polymer. US 2010/0041770 discloses a composite material formed by mixing a polymer phase with a solvent, adding a bioresorbable ceramic phase, and thereafter allowing the solvent to diffuse out of the polymer in the presence of water, to cause solidification of the polymer phase. The composite formed does not have initial porosity for rapid bone in-growth, though pores may form later by degradation of one of the phases.

US 2005/0251266 discloses a mouldable composite comprising ceramic granules coated with a biocompatible polymer and a plasticizer such that the polymer is initially deformable and then hardens upon removal of the plasticizer by placing in water. However, coating the granules is difficult and the specialist processes which need to be employed leads to an increase in cost. In addition, since all the granules are coated with polymer there is a delay in the osteoconductive effect of the bioceramic granules until at least some of the polymer degrades.

The present invention seeks to address at least some of these problems by providing a macroporous material for filling bone voids, which preferably includes one or more of the following characteristics: is mouldable/formable; sets to a hard and tough material; is able to bear loads; allows for rapid bone in-growth; and is biodegradable and substantially replaced by bone without substantially compromising the structural integrity of the site of application.

In its broadest sense the present invention provides a bone void filler comprising a bioresorbable granulated polymer and a biocompatible water-miscible solvent.

According to the present invention there is provided an implant material for bone void filling comprising bioresorbable polymer granules and a biocompatible water-miscible solvent, wherein the solvent at least partially dissolves and/or softens the polymer granules to form a mouldable mass that can be used to fill a bone defect, but which hardens when the implant material is exposed to water, and wherein the implant material has macroporosity suitable for bone in-growth.

Suitably, the implant material contains pores of between about 50 and 3000 microns; preferably 100 and 2000 microns; more preferably 120 and 1500 microns, which pores provide a macroporosity level suitable for bone in-growth.

Suitably, the implant material has an open porosity greater than 15%. Preferably, the implant material has an open porosity of between about 15%-70%; more preferably about 20%-55%; most preferably about 25%-45%.

Upon addition of the biocompatible water-miscible solvent to the bioresorbable polymer granules the granules soften and/or partially or fully dissolve causing them to become “sticky” and form a mouldable or flowable mass that can be delivered to the bone defect and which conforms to the shape of the defect. In the presence of water or an aqueous environment, such as being placed in the body, the solvent is removed and the implant material hardens into a mass with interconnected macroporosity.

Suitably, the bioresorbable polymer granules include particles, flakes or powder.

Suitably, the implant material further includes a bioceramic material. Suitably, the bioceramic material is formed as a mixture with the bioresorbable polymer. Preferably, the bioceramic material comprises granules, flakes or powder. In embodiments comprising a bioceramic powder, the powder may be dispersed within the bioresorbable polymer or bioresorbable polymer granules.

Preferably, the bioceramic material is porous. Suitably, the bioceramic material contains pores of between about 10 and 1000 microns; preferably 15 and 500 microns; more preferably 20 and 300 microns.

Suitably, the bioresorbable polymer granules include a core formed of a different material. Suitably, the core is formed from a second bioresorbable polymer which is different to the polymer of the bioresorbable polymer granules. Alternatively, the core is formed from a bioceramic material. Preferably, the bioceramic material is a bioceramic granule or powder. Optionally, the core includes an inner core and an outer core, wherein the inner core is formed from a bioceramic material and the outer core is formed from a second bioresorbable polymer. The core may also be formed from a bioresorbable polymer having a bioceramic powder dispersed therein. In such embodiments, the powder may be uniformly or non-uniformly dispersed.

Optionally, the implant material includes a bioactive or therapeutic agent. Suitably, the core includes a bioactive or therapeutic agent. Preferably, the outer core includes a bioactive or therapeutic agent.

Preferably, the bioactive or therapeutic agent includes at least one of: a growth factor such as any bone morphogenic protein (BMP), platelet derived growth factor (PDGF), growth hormone, transforming growth factor-beta (TGF-beta), insulin-like growth factor; a bisphosphonate such as alendronate, zoledronate; an antibiotic such as gentamicin, vancomycin, tobramicin; an anti-cancer drug such as paclitaxel, mercatopurine; an anti-inflammatory agent such as salicylic acid, indomethacine; an analgesic such as salicylic acid.

The bioactive or therapeutic agent may also be incorporated into the implant material by: coating onto the bioceramic granules; incorporating within the bioceramic granules; coating onto the polymer granules; incorporating within the polymer granules; incorporating within the biocompatible solvent; adding at the time of mixing the components or any combination of these methods to give a desired dispersion and release profile.

Preferably, the second bioresorbable polymer is less soluble in the biocompatible solvent than the first bioresorbable polymer. In this way, when the solvent is added, the surface of the bioresorbable polymer granules becomes softened and/or partially dissolves but the outer core layer, preferably containing a bioactive or therapeutic agent, remains largely intact. The bioactive or therapeutic agent will be released from the outer core layer as the first bioresorbable polymer is absorbed.

Optionally, the same or a different bioactive or therapeutic agent can be incorporated into the first bioresorbable polymer. Suitably, where the bioactive or therapeutic agents are the same they have different release rates according to the different release characteristics and/or degradation rates of the first and second bioresorbable polymers.

Preferably, the bioceramic granules include at least one of: calcium phosphate, including hydroxyapatite, any substituted hydroxyapatite (e.g. silicon, carbonate, magnesium, strontium, fluoride), tricalcium phosphate, biphasic calcium phosphate, tetracalcium phosphate, octacalcium phosphate, dicalcium phosphate dihydrate (brushite), dicalcium phosphate (monetite), calcium pyrophosphate, calcium pyrophosphate dihydrate, heptacalcium phosphate, calcium phosphate monohydrate; calcium sulphate; any bioactive glass (e.g. Bioglass) or glass ceramic (e.g. apatite-wollastonite); or any combination of these. The granules may be dense or porous.

Preferably, the first bioresorbable polymer includes at least one of: any polymer from the poly-alpha-hydroxyacid group, including poly(lactic acid), poly(glycolic acid), poly-L-lactide, poly-DL-lactide, poly(lactide-co-glycolide), poly(lactide-co-caprolactone), poly(L-lactide-co-DL-lactide), polycaprolactone; any bioresorbable polyanhydride, polyamide, polyorthoester, polydioxanone, polycarbonate, polyaminoacid, poly(amino-ester), poly(amido-carbonate), polyphosphazene, polyether, polyurethane, polycyanoacrylate, or any combination of these.

Preferably, the second bioresorbable polymer includes at least one of: a polymer from the poly-alpha-hydroxyacid group, including poly(lactic acid), poly(glycolic acid), poly-L-lactide, poly-DL-lactide, poly(lactide-co-glycolide), poly(lactide-co-caprolactone), poly(L-lactide-co-DL-lactide), polycaprolactone; any bioresorbable polyanhydride, polyamide, polyorthoester, polydioxanone, polycarbonate, polyaminoacid, poly(amino-ester), poly(amido-carbonate), polyphosphazene, polyether, polyurethane, polycyanoacrylate; a polysaccharide optionally including alginate, chitosan, carboxymethyl cellulose, hydroxypropylmethyl cellulose, dextran, hyaluronic acid, or any combination of these.

Preferably, the biocompatible, water miscible solvent includes at least one of: N-methyl-pyrollidone, dimethyl sulphoxide, acetone, poly(ethylene glycol), tetrahydrofuran, isopropanol, or caprolactone.

Optionally, the implant material includes a water soluble porogen that is not soluble in the biocompatible solvent. Preferably, the water soluble porogen includes at least one of: a soluble inorganic salt such as sodium chloride; any soluble organic compound such as sucrose; or a water soluble polymer such as poly(ethylene glycol), poly(vinyl alcohol), polysaccharide such as carboxymethylcellulose.

Compared with poly(methyl methacrylate) bone cements, aspects of the present invention are macroporous and fully bioresorbable.

Compared with bioceramic blocks, aspects of the present invention have the advantage of being injectable and/or mouldable and capable of conforming to irregular shaped bone defects.

Compared with bioceramic granules, aspects of the present invention have the advantage of hardening in-situ to form a cohesive mass, thus preventing the possibility of granules migrating. This could be particularly advantageous when the implant is being used to deliver a drug or therapeutic agent, particularly one which stimulates bone formation, such as BMP, as it reduces the possibility of bone forming in unwanted areas—particularly important if the implant is being used in areas such as the spine where there may be nerves etc near to the bone implant.

Compared with the implant material of US 2010/0041770, aspects of the invention described here have the advantage of having immediate connected macroporosity suitable for rapid bone in-growth.

Compared with the implant material of US 2005/0251266, aspects of the present invention keep at least some of the bioactive/therapeutic molecule within an intact coating layer which is not removed from the granules when the biocompatible solvent is added. This allows for better control and sustained release of the molecule. Also, in embodiments having more than one layer of polymer coating with different release and/or degradation profiles, the overall release of drug can be tailored or the system used to deliver different compounds with different release profiles. In addition, aspects of the invention do not require pre-coating of the ceramic granules, and furthermore, the fact that a portion of the granules comprise a bioresorbable polymer allows for the creation of greater porosity as the polymer granules degrade allowing more room for bone in-growth over time. In addition, we here disclose the use of water to modify the viscosity of the implant material prior to implantation in order to achieve the desired handling characteristics.

The viscosity of the implant material prior to hardening can be adjusted by the addition of water after the addition and mixing of the solvent. If an injectable/flowable material is desired then no water is added but by adding water prior to implantation a more putty-like/mouldable consistency can be achieved.

The above and other aspects of the invention will now be described with reference to the following drawings in which:

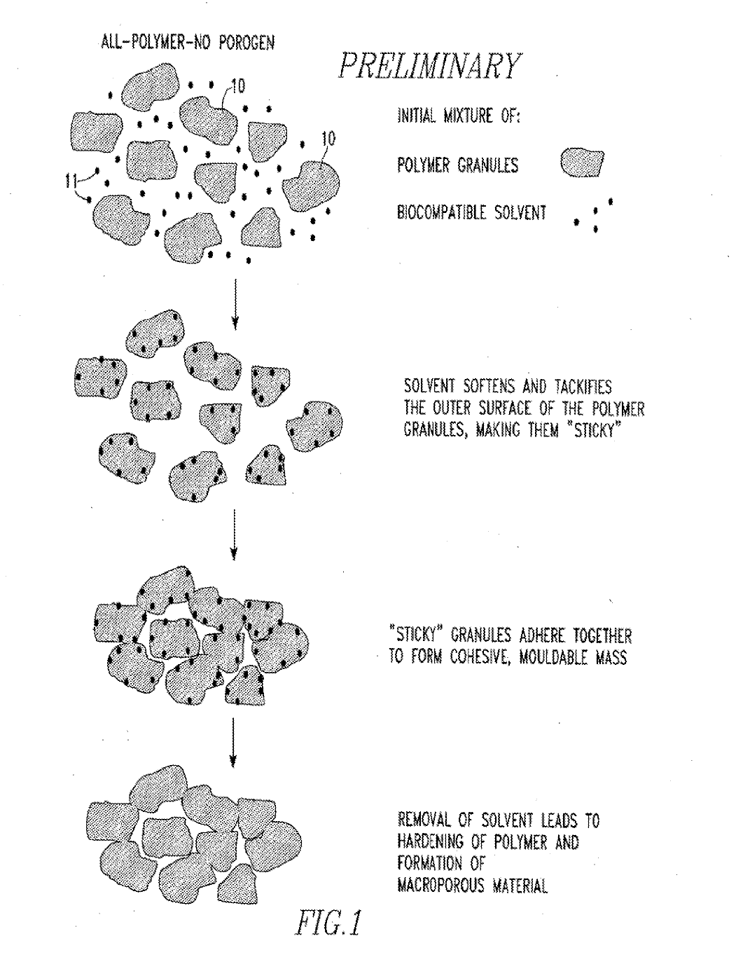

FIG. 1 is a schematic illustration of a first embodiment, according to the present invention, of an implant material for bone void filling;

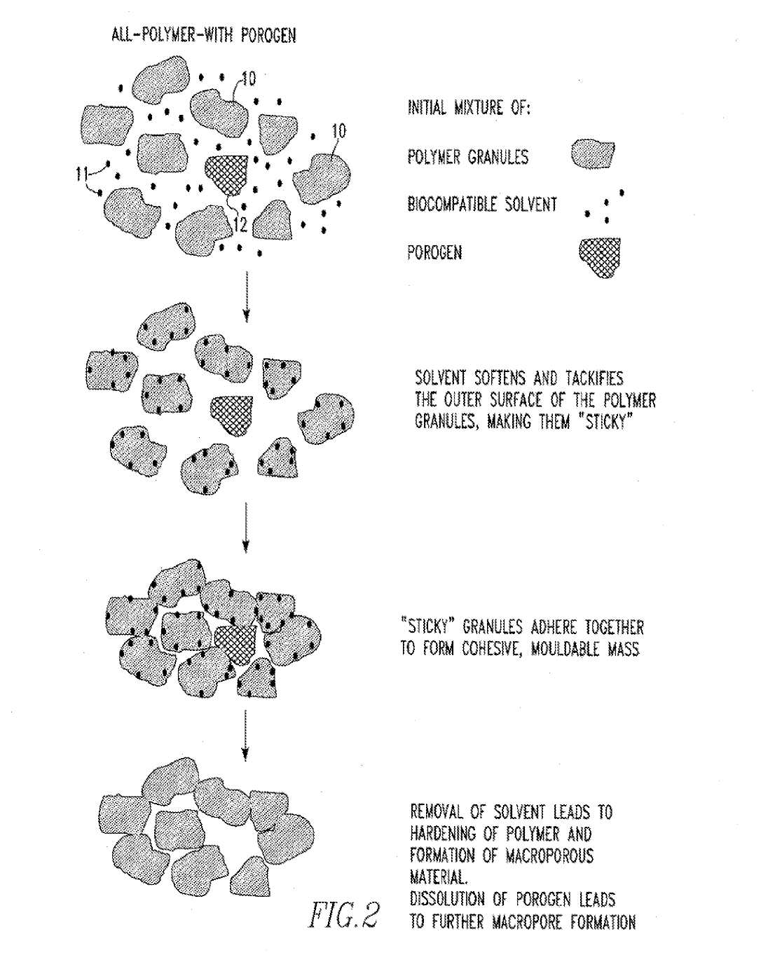

FIG. 2 is a schematic illustration of a second embodiment, according to the present invention, of an implant material for bone void filling;

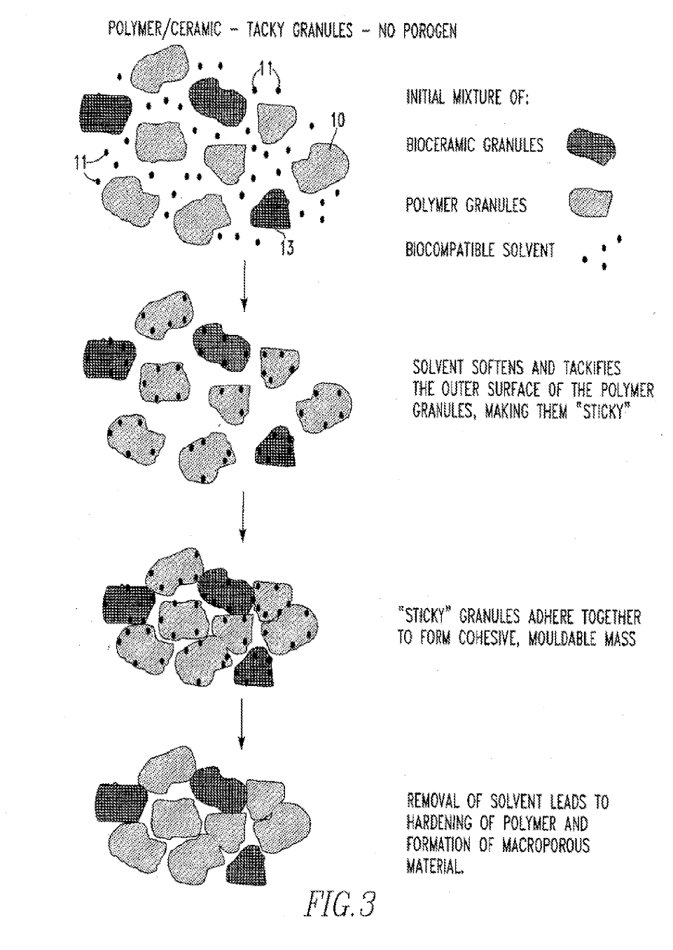

FIG. 3 is a schematic illustration of a third embodiment, according to the present invention, of an implant material for bone void filling;

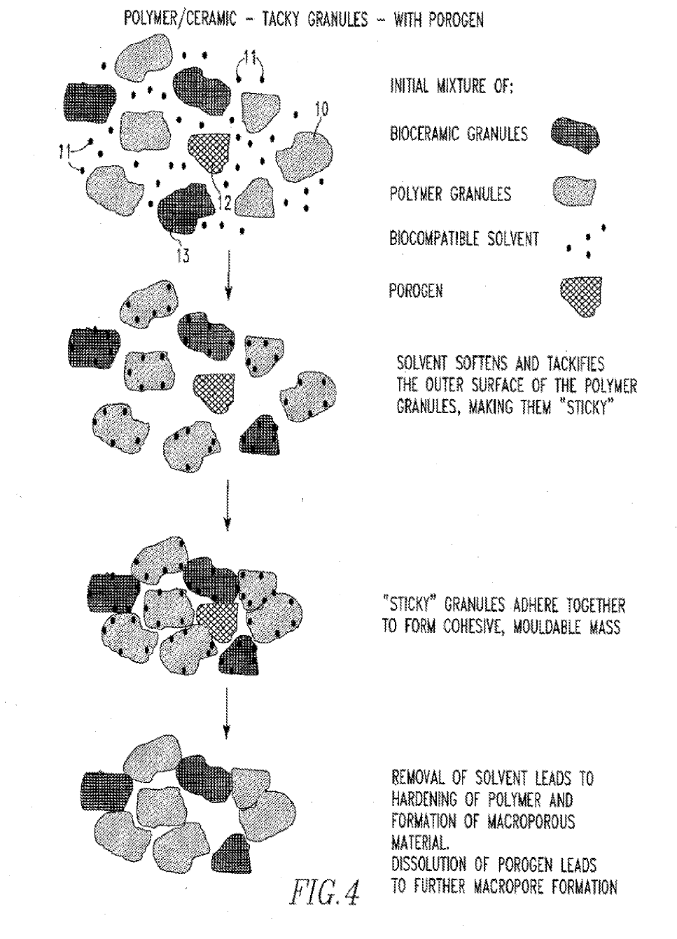

FIG. 4 is a schematic illustration of a fourth embodiment, according to the present invention, of an implant material for bone void filling;

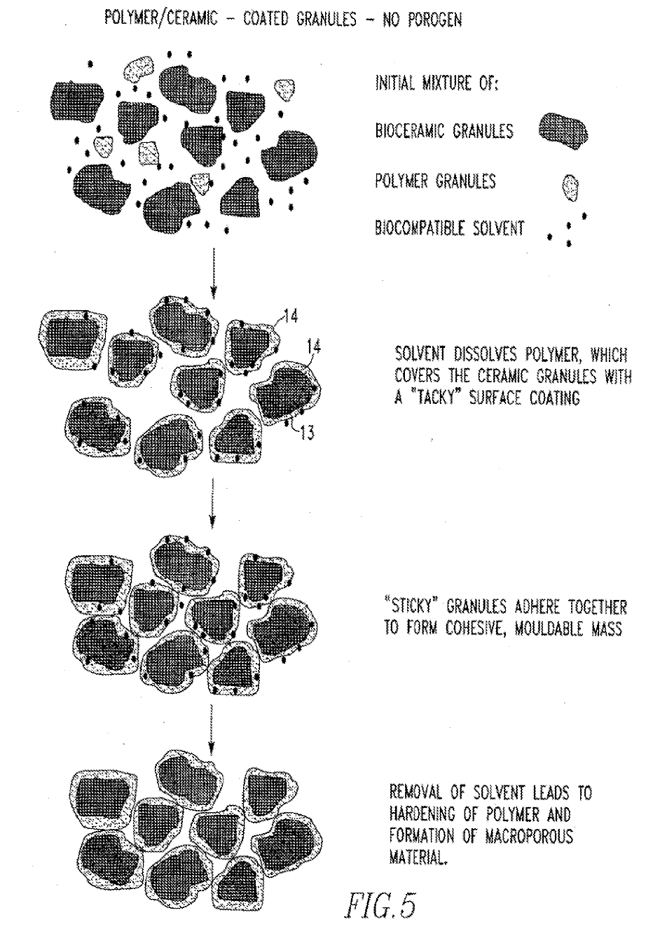

FIG. 5 is a schematic illustration of a fifth embodiment, according to the present invention, of an implant material for bone void filling;

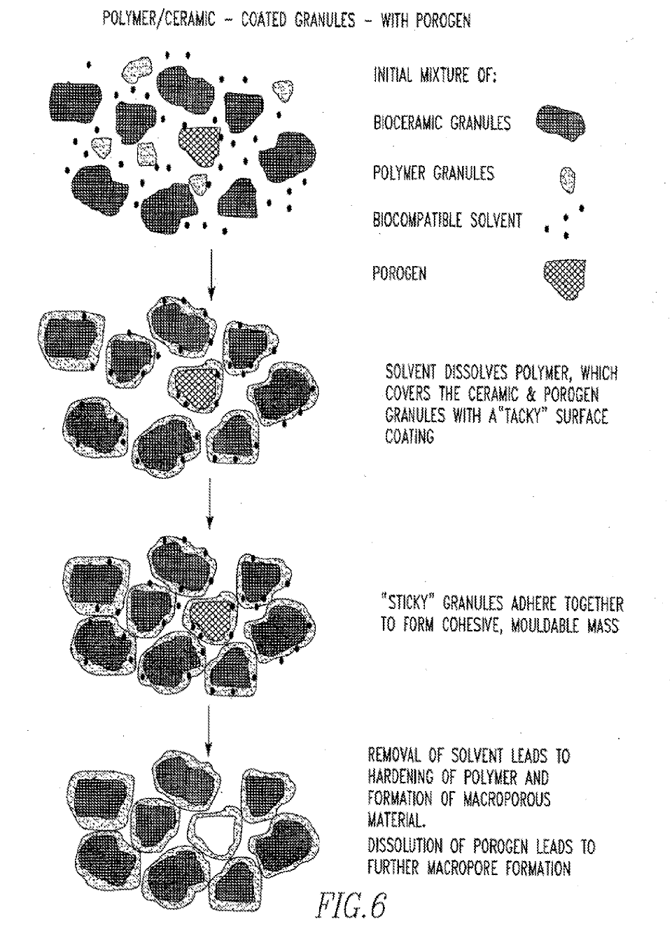

FIG. 6 is a schematic illustration of a sixth embodiment, according to the present invention, of an implant material for bone void filling;

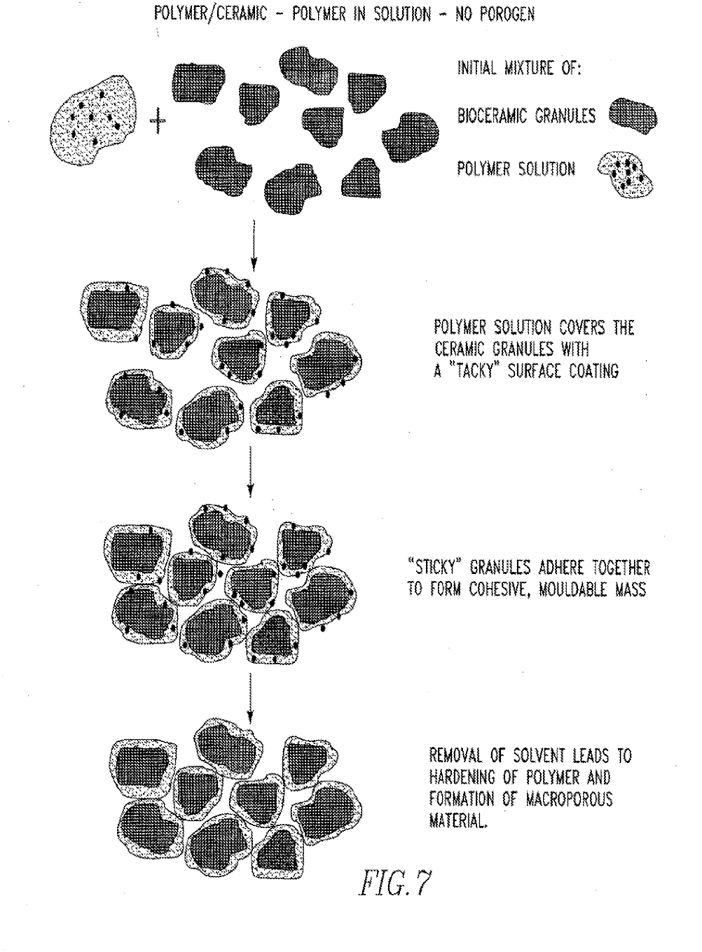

FIG. 7 is a schematic illustration of a seventh embodiment, according to the present invention, of an implant material for bone void filling;

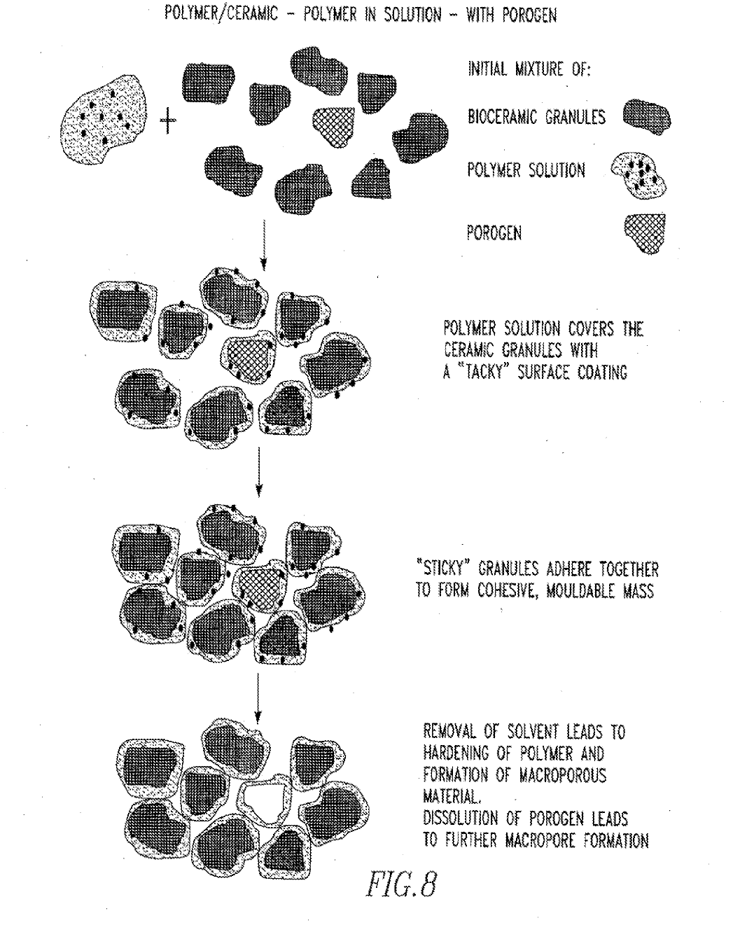

FIG. 8 is a schematic illustration of an eighth embodiment, according to the present invention, of an implant material for bone void filling;

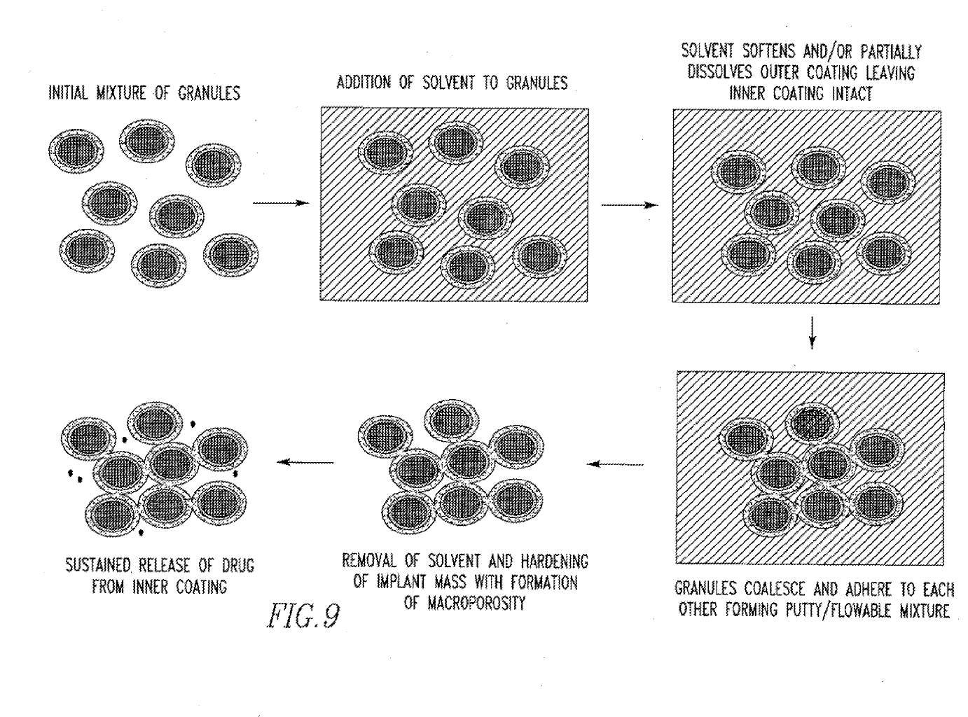

FIG. 9 is a schematic illustration of a ninth embodiment, according to the present invention, of an implant material for bone void filling; and

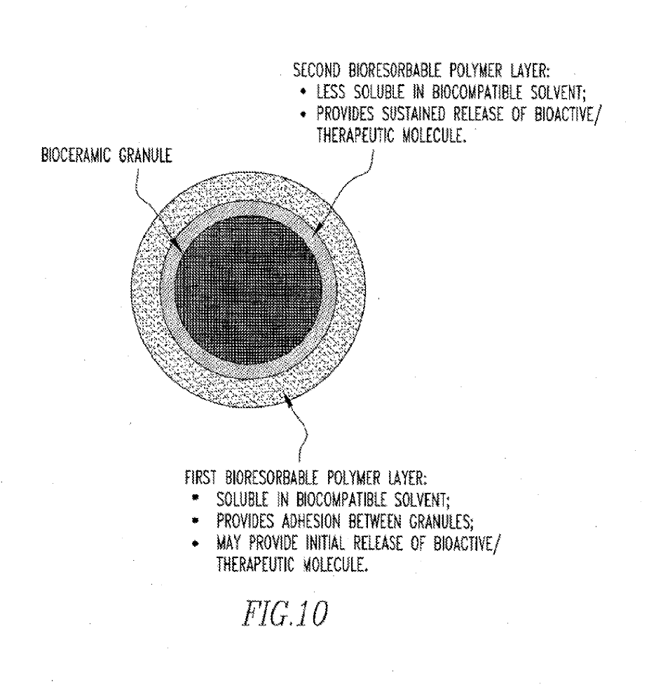

FIG. 10 is a close-up schematic view of the embodiment of FIG. 9.

Referring to FIG. 1, there is shown schematically an implant material precursor for bone void filling comprising polymer granules 10 and a biocompatible solvent 11. As the solvent 11 is mixed with the polymer granules the solvent softens and tackifies the outer surface of the polymer granules, giving them a ‘sticky’ character. In this state, the granules adhere together to form a cohesive, mouldable implant material. The implant material can then be used to fill bone voids and defects (not shown). The biocompatible solvent is preferably water-miscible. In the presence of water or an aqueous environment, such as being placed in the body, the solvent is removed and the implant material hardens into a mass with interconnected macroporosity. Consequently, the macroporous material allows for tissue ingrowth, particularly bone tissue ingrowth.

The polymer granules are formed from biosorbable materials such as poly(lactic acid), poly(glycolic acid), poly-L-lactide, poly-DL-lactide, poly(lactide-co-glycolide), poly(lactide-co-caprolactone), poly(L-lactide-co-DL-lactide), polycaprolactone; any bioresorbable polyanhydride, polyamide, polyorthoester, polydioxanone, polycarbonate, polyaminoacid, poly(amino-ester), poly(amido-carbonate), polyphosphazene, polyether, polyurethane, polycyanoacrylate, or any combination of these, and as the polymer degrades and is absorbed by the body new bone forms and advances to replace substantially all of the polymer material.

The biocompatible water miscible solvent may be selected from: N-methyl-pyrollidone, dimethyl sulphoxide, acetone, poly(ethylene glycol), tetrahydrofuran, isopropanol, or caprolactone.

As shown in FIGS. 2 and 4, a porogen 12 can be incorporated in the implant material leading to the formation of further macropores within the set composition. Typically, the porogen will be a soluble inorganic salt such as sodium chloride; a soluble organic compound such as sucrose; or a water soluble polymer such as poly(ethylene glycol), poly(vinyl alcohol), polysaccharide such as carboxymethylcellulose.

The implant material may also include a bioceramic material in the form of granules 13, as illustrated in FIG. 3. The bioceramic material may be at least one of: calcium phosphate, including hydroxyapatite, a substituted hydroxyapatite (e.g. silicon, carbonate, magnesium, strontium, fluoride), tricalcium phosphate, biphasic calcium phosphate, tetracalcium phosphate, octacalcium phosphate, dicalcium phosphate dihydrate (brushite), dicalcium phosphate (monetite), calcium pyrophosphate, calcium pyrophosphate dihydrate, heptacalcium phosphate, calcium phosphate monohydrate; calcium sulphate; a bioactive glass or glass ceramic; or any combination of these.

According to this embodiment, the solvent softens and tackifies the outer surface of the polymer granules, making them sticky. The granules then adhere to each other and also the bioceramic granules, and as the solvent is removed, the polymer hardens and incorporates the bioceramic granules in the set macroporous structure. The bioceramic granules add strength and rigidity to the implant material, and are osteoconductive to encourage bone in-growth. Further, because only the outer surface of the polymer granules is softened, the polymer does not spread to coat the surface of the bioceramic granules, and therefore much of the outer surface of the bioceramic granules remains exposed. Accordingly, there is substantially no delay to initiation of the osteoconductive effect.

In further alternative embodiments shown in FIGS. 5 and 6, the biocompatible solvent fully dissolves the polymer granules in the presence of the bioceramic granules and forms a coating 14 over each surface. This can be achieved in the presence or absence of a porogen. Alternatively, a similar result can be achieved by pre-mixing the solvent and polymer and then adding the bioceramic granules, and optionally a porogen, to this mixture in order to form the implant material (FIGS. 7 and 8).

The implant material may also include a bioactive or therapeutic agent. Examples of such include, but are not limited to a growth factor such as a bone morphogenic protein (BMP), platelet derived growth factor (PDGF), growth hormone, transforming growth factor-beta (TGF-beta), insulin-like growth factor; a bisphosphonate such as alendronate, zoledronate; an antibiotic such as gentamicin, vancomycin, tobramicin; an anti-cancer drug such as paclitaxel, mercatopurine; an anti-inflammatory agent such as salicylic acid, indomethacine; or an analgesic such as salicylic acid.

In further embodiments of the invention, shown in FIGS. 9 and 10, the bioresorbable polymer granules include a core formed of a different material. The core material may be a different bioresorbable polymer, having different properties to the first bioresorbable polymer granules, or may be a bioceramic material. In embodiments which incorporate a bioceramic core, the material will be a bioceramic granule or powder. In further embodiments, the core includes an inner core and an outer core, where the inner core is formed from a bioceramic material and the outer core is formed from a second bioresorbable polymer.

Referring to FIG. 10, there is shown a polymer granule formed from a first bioresorbable polymer. The polymer granule includes a core having an inner core formed from a bioceramic material, and an outer core, formed from a second bioresorbable polymer. The first bioresorbable polymer will be at least partially soluble in the biocompatible solvent so that it provides adhesion between granules. If the first bioresorbable polymer includes a bioactive or therapeutic agent, it may provide an initial release of that agent as the polymer starts to degrade and be absorbed.

The second bioresorbable polymer may be: a polymer comprising a poly-alpha-hydroxyacid group, including poly(lactic acid), poly(glycolic acid), poly-L-lactide, poly-DL-lactide, poly(lactide-co-glycolide), poly(lactide-co-caprolactone), poly(L-lactide-co-DL-lactide), polycaprolactone; any bioresorbable polyanhydride, polyamide, polyorthoester, polydioxanone, polycarbonate, polyaminoacid, poly(amino-ester), poly(amido-carbonate), polyphosphazene, polyether, polyurethane, polycyanoacrylate; a polysaccharide comprising alginate, chitosan, carboxymethyl cellulose, hydroxypropylmethyl cellulose, dextran, hyaluronic acid, or any combination of these. The second bioresorbable polymer is generally less soluble in the biocompatible solvent. Where bioactive or therapeutic agents are incorporated in the second bioresorbable polymer, this allows for a sustained release of said agent.

### EXAMPLES

### Example 1

Materials: β-tricalcium phosphate granules (GenOs 1-2 mm, supplied by Orthos Ltd); poly-DL-lactide-co-glycolide (PDLGA) 85:15 (Puresorb, supplied by Purac); N-methyl-pyrollidone (NMP) (supplied by Sigma-Aldrich). Prior to use the PDLGA raw granules were reduced in particle size by cryo-milling for a total of about 6 minutes to a final particle size <1 mm.

Method: 1 ml TCP granules was mixed with 1 ml PDLGA 85:15 granules. 0.5 ml of NMP was added and the mixture was stirred and kneaded with a spatula until it formed a putty-like consistency. The mass could be moulded in the hands. It was placed in a cylindrical plastic mould (internal diameter=11.8 mm) and packed using finger pressure. The material was then pushed out of the mould and was seen to maintain its shape. It was placed in deionized water at room temperature. After approximately 5 minutes the sample was removed and had hardened sufficiently that it was no longer mouldable. It was observed that the material had maintained porosity between the fused granules.

The sample was stored overnight in deionized water at 37° C. After 24 hours the cylindrical samples were all cut to a height of 1.5 cm and tested in compression using an Instron 5569 Universal Testing Machine at a rate of 5 mm/min.

Compression testing gave a yield stress of 5.5 MPa. There was no peak in the stress-strain curve indicating a tough material.

### Example 2

Example 1 was repeated but this time 1 ml of NMP was added. In this case the polymer granules fully dissolved and a solid plug was formed with less visible porosity. The sample was stored in deionized water at 37° C. and tested in compression as described in Example 1. Compression testing gave a yield stress of 4 MPa. There was no peak in the stress-strain curve indicating a tough material.

### Example 3

1 ml of TCP was mixed with 0.25 ml of PDLGA 85:15. 1 ml of NMP was then added and stirred to dissolve the polymer. The mixture in this case was flowable and less putty-like than the previous examples. However, when 1 ml of water was added to the mass it immediately became more cohesive and putty-like. It was packed into the mould using finger pressure as before and then pushed out into deionized water. After about 5 minutes the sample was removed and the plug had hardened. It appeared more porous than examples 1 and 2. The sample was stored in deionized water at 37° C. and tested in compression as described in Example 1. Compression testing gave a peak stress of 1 MPa.

### Example 4

0.5 ml of TCP was mixed with 0.5 ml sucrose (granulated—supplied by Sigma-Aldrich, Product Code 84097) and 0.5 ml PDLGA 85:15. 0.5 ml NMP was added to the mixture and stirred and kneaded with a spatula to form a putty. Again the mixture was packed into the mould then pushed out into water. After about 5 minutes the sample was removed and examined and seen to have hardened. Pores were visible between the granules and also from the dissolution of the sucrose. The sample was stored in deionized water at 37° C. and tested in compression as described in Example 1. The sample gradually collapsed under compression and no yield point or peak stress was visible on the stress-strain curve.

### Example 5

0.5 ml TCP was mixed with 0.5 ml sucrose and 0.25 ml PDLGA 85:15. 1 ml of NMP was added. As for Example 3, a flowable system was formed. 0.5 ml water was added and this caused the mixture to form a putty-like consistency. Again it was packed into the mould and pushed out into water. After about 5 minutes the sample was examined and seen to have hardened. Pores were visible between the granules and also from the dissolution of the sucrose. The sample was stored in deionized water at 37° C. and tested in compression as described in Example 1. The sample gradually collapsed under compression and no yield point or peak stress was visible on the stress-strain curve.

### Example 6

1 ml TCP was mixed with 0.5 ml powdered PDLGA 50:50 (supplied by Aldrich (The PLGA was not cryo-milled as it was already in powdered form). 0.2 ml NMP was added dropwise to the dry constituents and thoroughly mixed by hand with a spatula to form a loosely cohesive mass. Five drops of deionised water were then added with further mixing to produce a mouldable putty. This was packed and compressed into the mould and then pushed out into deionised water. The sample quickly hardened to form a porous cylindrical plug. The sample was stored in deionized water at 37° C. and tested in compression as described in Example 1. Compression testing gave a peak stress of 0.5 MPa.

### Example 7

1 ml TCP was mixed with 0.2 ml powdered PDLGA (50:50). 0.15 ml NMP was added dropwise to the dry constituents and thoroughly mixed by hand with a spatula to form a loosely cohesive mass. Five drops of deionised water were then added with further mixing to produce a mouldable putty. This was packed and compressed into the mould and then pushed out into deionised water. The sample quickly hardened to form a porous cylindrical plug. The sample was stored in deionized water at 37° C. and tested in compression as described in Example 1. Compression testing gave a peak stress of 1.25 MPa.

### Example 8

1 ml TCP was mixed with 0.1 ml powdered PDLGA (50:50). 0.15 ml NMP was added dropwise to the dry constituents and thoroughly mixed by hand with a spatula to form a loosely cohesive mass. Five drops of deionised water were then added with further mixing to produce a mouldable putty. This was packed and compressed into the mould and then pushed out into deionised water. The sample quickly hardened to form a porous cylindrical plug. The sample was too friable to undergo compression testing.

### Example 9

0.5 ml TCP granules were combined with 0.5 ml hydroxyapatite granules (2-3 mm—supplied by Plasma Biotal Ltd)) and 0.2 ml powdered PDLGA (50:50). 0.25 ml NMP was added dropwise to the dry mixture and thoroughly mixed with a spatula. The further addition of 5 drops of deionised water produced a cohesive putty that was packed into the mould and then released out into deionised water. The sample quickly hardened to form a porous cylindrical plug. The sample was too friable to undergo compression testing.

### Example 10

0.4 ml TCP granules were combined with 1.2 ml hydroxyapatite granules (2-3 mm) and 0.8 ml powdered PDLGA (50:50). 0.25 ml NMP was added dropwise to the dry mixture and thoroughly mixed with a spatula. The further addition of 5 drops of deionised water produced a cohesive putty that was packed into the mould and then released out into deionised water. The sample quickly hardened to form a porous cylindrical plug. The sample was stored in deionized water at 37° C. and tested in compression as described in Example 1. Compression testing gave a peak stress of 0.9 MPa.

### Example 11

1 ml of TCP was mixed with 0.25 ml of PDLGA (85:15). 1 ml of ε-caprolactone (supplied by Acros Organics) was then added and stirred to form a flowable mass. The material was packed into the mould and 1 ml of water was added. The plug could then be pushed out of the mould into deionized water. After about 5 minutes the sample was removed and examined. It was a cohesive porous cylinder but still quite soft; it had fully hardened after 16 hours. The sample was stored in deionized water at 37° C. and tested in compression as described in Example 1. Compression testing gave a peak stress of 0.8 MPa.

### Example 12

1 ml TCP was mixed with 0.25 ml PDLGA 85:15 and then 0.98 g NMP was added. The mixture was stirred to form a mouldable mass and was then packed into a cylindrical mould (internal diameter=8.5 mm) and compressed using finger pressure. The plug of material was pushed out into deionized water and was seen to harden instantly on contact with the water.

The sample was stored in deionized water for 24 hours and then removed and air dried.

The sample was prepared for Micro-CT analysis by mounting the bone void filler specimen directly onto a brass pin sample holder using an adhesive tab on the base of the bone void filler. Micro-CT images were acquired on a Skyscan 1173 Micro-CT using a micro focused X-ray source with a voltage of 85 kV and a current of 68 μA. X-ray shadow images were acquired with a 0.4 deg step size over a 180 deg acquisition angle, with 4 averages and 6 μm resolution. The X-ray shadow images were reconstructed into a stack of 2D cross-sections using a reconstruction program (N-Recon) supplied by Skyscan. The Micro-CT images were reconstructed using a smoothing factor of 2, a ring artefact correction of 12 and a beam hardness correction factor between 50%-65%.

The results from the micro-CT scanning were as follows:

| | |
|---|---|
| % Open Pore Space | 41.0 |
| % Closed Pore Space | 0.9 |
| Total % Pore Space | 41.9 |
| % Material | 58.1 |

The sample was then tested in compression using an Instron 5569 Universal testing Machine at a rate of 2.5 mm/min. The sample had a compressive modulus of 2.33 MPa and a failure stress of 0.13 MPa.

### Example 13

1 ml TCP was mixed with 1 ml PDLGA 85:15 and then 0.5 g NMP was added. The mixture was stirred to form a mouldable mass and was then packed into a cylindrical mould (internal diameter=8.5 mm) and compressed using finger pressure. The plug of material was pushed out into deionized water and was seen to harden instantly on contact with the water.

The sample was stored in deionized water for 24 hours and then removed and air dried.

The sample was analysed by micro-CT as described in Example 12. The results from the micro-CT scanning were as follows:

| | |
|---|---|
| % Open Pore Space | 19.8 |
| % Closed Pore Space | 1.6 |
| Total % Pore Space | 21.4 |
| % Material | 78.6 |

The sample was then tested in compression as described in Example 12. The sample had a compressive modulus of 77.0 MPa and a failure stress of 3.78 MPa.

### Example 14

2 ml (=2.13 g) HA granules (2-3 mm, Plasma Biotal) was mixed with 0.24 g PDLGA 85:15 and then 0.49 g NMP was added. The mixture was stirred to dissolve the polymer and coat the ceramic particles. This formed a mouldable mass which was then packed into a cylindrical mould (internal diameter=11.8 mm) and compressed using finger pressure. The plug of material was pushed out into deionized water and was seen to harden instantly on contact with the water.

The sample was stored in deionized water for 24 hours and then removed and air dried.

The sample was analysed by micro-CT as described in Example 12. The results from the micro-CT scanning were as follows:

| | |
|---|---|
| % Open Pore Space | 41.8 |
| % Closed Pore Space | 0.1 |
| Total % Pore Space | 41.9 |
| % Material | 58.1 |

The sample was then tested in compression as described in Example 12. The sample had a compressive modulus of 1.92 MPa and a failure stress of 0.14 MPa.

### Example 15

0.19 g PDLGA 85:15 was mixed with 0.37 g NMP to dissolve the polymer. 2 ml (=2.12 g) HA granules (2-3 mm, Plasma Biotal) was then mixed into the polymer solution. The mixture was stirred coat the ceramic particles. This formed a mouldable mass which was then packed into a cylindrical mould (internal diameter=11.8 mm) and compressed using finger pressure. The plug of material was pushed out into deionized water and was seen to harden instantly on contact with the water.

The sample was stored in deionized water for 24 hours and then removed and air dried.

The sample was analysed by micro-CT as described in Example 12. The results from the micro-CT scanning were as follows:

| | |
|---|---|
| % Open Pore Space | 39.7 |
| % Closed Pore Space | 0.1 |
| Total % Pore Space | 39.8 |
| % Material | 60.2 |

The sample was then tested in compression as described in Example 12. The sample had a compressive modulus 3.21 and a failure stress of 0.18 MPa.

### Example 16

A 33.3% w/w solution of PLGA 85:15 in NMP was prepared by mixing 3 g PDLGA with 6 g NMP and allowing to stand overnight at room temperature until the polymer was fully dissolved.

4 ml (=1.46 g) HA/TCP granules (0.8-1.5 mm, supplied by Ceramisys Ltd) was mixed with 0.52 g of the 33.3% PDLGA solution and stirred thoroughly to coat the granules. The resulting mass was packed into a cylindrical mould (internal diameter=11.8 mm) and compressed using finger pressure. The plug of material was pushed out into deionized water and was seen to harden instantly on contact with the water.

The sample was stored in deionized water for 3 days and then removed and air dried.

The sample was analysed by micro-CT as described in Example 12. The results from the micro-CT scanning were as follows:

| | |
|---|---|
| % Open Pore Space | 44.5 |
| % Closed Pore Space | 0.4 |
| Total % Pore Space | 44.9 |
| % Material | 55.1 |

The sample was then tested in compression as described in Example 12. The sample had a compressive modulus of 3.96 MPa and a failure stress of 0.24 MPa.

### Example 17

2 ml (=2.18 g) HA granules (2-3 mm, plasma Biotal) was mixed with 0.53 g of the 33.3% PDLGA solution used in Example 16 and stirred thoroughly to coat the granules. The resulting mass was packed into a cylindrical mould (internal diameter=11.8 mm) and compressed using finger pressure. The plug of material was pushed out into deionized water and was seen to harden instantly on contact with the water.

The sample was stored in deionized water for 3 days and then removed and air dried.

The sample was analysed by micro-CT as described in Example 12. The results from the micro-CT scanning were as follows:

| | |
|---|---|
| % Open Pore Space | 42.9 |
| % Closed Pore Space | 0.0 |
| Total % Pore Space | 42.9 |
| % Material | 57.1 |

The sample was then tested in compression as described in Example 12. The sample had a compressive modulus of 13.3 MPa and a failure stress of 0.37 MPa.

### Example 18

2 ml (=2.12 g) HA granules (2-3 mm, plasma Biotal) was mixed with 0.70 g of the 33.3% PDLGA solution used in Example 16 and stirred thoroughly to coat the granules. The resulting mass was packed into a cylindrical mould (internal diameter=11.8 mm) and compressed using finger pressure. The plug of material was pushed out into deionized water and was seen to harden instantly on contact with the water.

The sample was stored in deionized water for 24 hours and then removed and air dried.

The sample was analysed by micro-CT as described in Example 12. The results from the micro-CT scanning were as follows:

| | |
|---|---|
| % Open Pore Space | 36.0 |
| % Closed Pore Space | 0.1 |
| Total % Pore Space | 36.1 |
| % Material | 63.9 |

The sample was then tested in compression as described in Example 12. The sample had a compressive modulus of 31.5 MPa and a failure stress of 1.52 MPa.

### Example 19

2 ml (=0.72 g) HA/TCP granules (0.8-1.5 mm, Ceramisys) was mixed with 2 ml (=1.72 g) sucrose, then further mixed with 0.70 g of the 33.3% PDLGA solution used in Example 16 and stirred thoroughly to coat the granules. The resulting mass was packed into a cylindrical mould (internal diameter=11.8 mm) and compressed using finger pressure. The plug of material was pushed out into deionized water and was seen to harden instantly on contact with the water but with some shedding of ceramic particles. The sucrose was also seen to dissolve, creating pores.

The sample was stored in deionized water for 24 hours and then removed and air dried.

The sample was analysed by micro-CT as described in Example 12. The results from the micro-CT scanning were as follows:

| | |
|---|---|
| % Open Pore Space | 55.3 |
| % Closed Pore Space | 0.5 |
| Total % Pore Space | 55.8 |
| % Material | 44.2 |

The sample was then tested in compression as described in Example 12. The sample had a compressive modulus of 6.07 MPa and a failure stress of 2.13 MPa.

### Example 20

2.5 ml PDLGA 85:15 granules (as received—not cryo-milled) was mixed with 0.32 g NMP. The NMP made the polymer granules tacky so that a mouldable cohesive mass was formed. This was packed into a cylindrical mould (internal diameter=11.8 mm) and compressed using finger pressure. The plug of material was pushed out into deionized water and was seen to harden instantly on contact with the water.

The sample was stored in deionized water for 24 hours and then removed and air dried.

The sample was analysed by micro-CT as described in Example 12. The results from the micro-CT scanning were as follows:

| | |
|---|---|
| % Open Pore Space | 37.7 |
| % Closed Pore Space | 0.2 |
| Total % Pore Space | 37.9 |
| % Material | 62.1 |

The sample was then tested in compression as described in Example 12. The sample had a compressive modulus of 26.5 MPa and a failure stress of 3.65 MPa.

### Example 21

1.25 ml PDLGA 85:15 granules (as received—not cryo-milled) was mixed with 1.25 ml HA/TCP (0.8-1.5 mm, Ceramisys) and further mixed with 0.33 g NMP. The NMP made the polymer granules tacky so that a mouldable cohesive mass was formed. This was packed into a cylindrical mould (internal diameter=11.8 mm) and compressed using finger pressure. The plug of material was pushed out into deionized water and was seen to harden instantly on contact with the water. There was some shedding of ceramic particles from the surface when the plug was dispensed into water.

The sample was stored in deionized water for 24 hours and then removed and air dried.

The sample was analysed by micro-CT as described in Example 12. The results from the micro-CT scanning were as follows:

| | |
|---|---|
| % Open Pore Space | 42.6 |
| % Closed Pore Space | 1.2 |
| Total % Pore Space | 43.8 |
| % Material | 56.2 |

The sample was then tested in compression as described in Example 12. The sample had a compressive modulus of 0.97 MPa and a failure stress of 0.16 MPa.

Table 1 below summarises the compositions and results from Examples 1-11

| | | | | | | | | | | Compression Test | |
|---|---|---|---|---|---|---|---|---|---|---|---|
| | | | | | | | | | | Load at | Peak |
| Example | | PDLGA | Porogen | | Ceramic | PDLGA | Solvent | Porogen | | Yield | Load |
| No. | Ceramic Type | Type | Type | Solvent | Vol. (ml) | Vol. (ml) | Vol. (ml) | Vol. (ml) | Water | (MPa) | (MPa) |
| 1 | TCP Porous | 85:15 | None | NMP | 1 | 1 | 0.5 | 0 | None | 5.5 | NA |
| | Granules (1-2 mm) | | | | | | | | | | |
| 2 | TCP Porous | 85:15 | None | NMP | 1 | 1 | 1 | 0 | None | 4 | NA |
| | Granules (1-2 mm) | | | | | | | | | | |
| 3 | TCP Porous | 85:15 | None | NMP | 1 | 0.25 | 1 | 0 | None | N/A | 1 |
| | Granules (1-2 mm) | | | | | | | | | | |
| 4 | TCP Porous | 85:15 | Sucrose | NMP | 0.5 | 0.5 | 0.5 | 0.5 | None | N/A | N/A |
| | Granules (1-2 mm) | | | | | | | | | | |
| 5 | TCP Porous | 85:15 | Sucrose | NMP | 0.5 | 0.25 | 1 | 0.5 | 0.5 ml | N/A | N/A |
| | Granules (1-2 mm) | | | | | | | | | | |
| 6 | TCP Porous | 50:50 | None | NMP | 1 | 0.5 | 0.2 | 0 | 5 drops | N/A | 0.5 |
| | Granules (1-2 mm) | | | | | | | | | | |
| 7 | TCP Porous | 50:50 | None | NMP | 1 | 0.2 | 0.15 | 0 | 5 drops | N/A | 1.25 |
| | Granules (1-2 mm) | | | | | | | | | | |
| 8 | TCP Porous | 50:50 | None | NMP | 1 | 0.1 | 0.15 | 0 | 5 drops | Not tested | |
| | Granules (1-2 mm) | | | | | | | | | | |
| 9 | TCP Porous | 50:50 | None | NMP | 1 | 0.2 | 0.25 | 0 | 5 drops | Not tested | |
| | Granules: HA Dense | | | | | | | | | | |
| | granules 50:50 | | | | | | | | | | |
| 10 | TCP Porous | 50:50 | None | NMP | 1.6 | 0.8 | 0.25 | 0 | 5 drops | N/A | 0.9 |
| | Granules: HA Dense | | | | | | | | | | |
| | granules 25:75 | | | | | | | | | | |
| 11 | TCP Porous | 85:15 | None | Caprolactone | 1 | 0.25 | 1 | 0 | None | N/A | 0.8 |
| | Granules (1-2 mm) | | | | | | | | | | |

Table 2 below summarises the compositions from Examples 12-21.

| | | | | | | | | Porogen |
|---|---|---|---|---|---|---|---|---|
| Example | | | Ceramic | Ceramic | PDLGA | PDLGA | NMP | (Sucrose) |
| No | Ceramic Type | PDLGA Type | Vol (ml) | Mass (g) | Vol (ml) | mass (g) | mass (g) | mass (g) |
| 12 | TCP Porous | Cryo-milled | 1 | | 0.25 | | 0.98 | 0 |
| | Granules (1-2 mm) | | | | | | | |
| 13 | TCP Porous | Cryo-milled | 1 | | 1 | | 0.50 | 0 |
| | Granules (1-2 mm) | | | | | | | |
| 14 | HA Dense | Cryo-milled | 2 | 2.13 | | 0.24 | 0.49 | 0 |
| | Granules (2-3 mm) | | | | | | | |
| 15 | HA Dense | Cryo-milled | 2 | 2.12 | | 0.19 | 0.37 | 0 |
| | Granules (2-3 mm) | | | | | | | |
| 16 | HA/TCP Porous | Solution | 4 | 1.46 | | 0.17 | 0.35 | 0 |
| | Granules (0.8-1.5 mm) | | | | | | | |
| 17 | HA Dense | Solution | 2 | 2.18 | | 0.18 | 0.35 | 0 |
| | Granules (2-3 mm) | | | | | | | |
| 18 | HA Dense | Solution | 2 | 2.12 | | 0.23 | 0.47 | 0 |
| | Granules (2-3 mm) | | | | | | | |
| 19 | HA/TCP Porous | Solution | 2 | 0.72 | | 0.23 | 0.47 | 1.72 |
| | Granules (0.8-1.5 mm) | | | | | | | |
| 20 | None | As received granules | 0 | | 2.5 | | 0.32 | 0 |
| 21 | HA/TCP Porous | As received granules | 1.25 | | 1.25 | | 0.33 | 0 |
| | Granules (0.8-1.5 mm) | | | | | | | |

Table 3 below summarises the testing results from Examples 12-21.

| | % Open | % Closed | Total % | | | Stress at |
|---|---|---|---|---|---|---|
| Example | Pore | Pore | Pore | % | Modulus | Failure |
| No | Space | Space | Space | Material | (MPa) | (MPa) |
| 12 | 41.0 | 0.9 | 41.9 | 58.1 | 2.33 | 0.13 |
| 13 | 19.8 | 1.6 | 21.4 | 78.6 | 77.00 | 3.78 |
| 14 | 41.8 | 0.1 | 41.9 | 58.1 | 1.92 | 0.14 |
| 15 | 39.7 | 0.1 | 39.8 | 60.2 | 3.21 | 0.18 |
| 16 | 44.5 | 0.4 | 44.9 | 55.1 | 3.96 | 0.24 |
| 17 | 42.9 | 0.0 | 42.9 | 57.1 | 13.30 | 0.37 |
| 18 | 36.0 | 0.1 | 36.1 | 63.9 | 31.50 | 1.52 |
| 19 | 55.3 | 0.5 | 55.8 | 44.2 | 6.07 | 2.13 |
| 20 | 37.7 | 0.2 | 37.9 | 62.1 | 26.50 | 3.65 |
| 21 | 42.6 | 1.2 | 43.8 | 56.2 | 0.97 | 0.16 |

The results in tables 1 and 3 show that materials able to withstand stresses up to 5 MPa or higher are achievable, while still maintaining a high level of porosity. The compressive strength of cancellous bone is typically in the range 2-12 MPa so it can be seen that it is possible to make bone void filling materials with strengths in this range (Examples 1, 2, 13, 19 and 20). The Young's modulus of cancellous bone is typically in the range 4-350 MPa and it can also be seen that it is possible to make materials with compressive moduli in this range (Examples 13, 16, 17, 18, 19, 20). All the samples had a high degree of porosity (20-60%) as seen in Table 3 and importantly most of this is interconnected porosity with only very low levels of closed pore space, thus allowing bone in-growth throughout the material. Inclusion of a porogen (such as in Example 19) can be seen to increase the porosity.

What is claimed is:

1. An implant material for bone void filling comprising bioresorbable polymer granules and a biocompatible water-miscible solvent, wherein the solvent at least partially dissolves and/or softens the polymer granules to form a moldable mass that can be used to fill a bone defect but which hardens when the implant material is exposed to water, and wherein the implant material has macroporosity suitable for bone in-growth.

2. An implant material according to claim 1 comprising pores of between about 50 and 3000 microns; preferably 100 and 2000 microns; more preferably 120 and 1500 microns, which pores provide a macroporosity level suitable for bone in-growth.

3. An implant material according to claim 1, wherein the bioresorbable polymer granules comprise particles, flakes or powder.

4. An implant material according to claim 1, wherein the implant material further comprises a bioceramic material.

5. An implant material according to claim 4, wherein the bioceramic material is formed as a mixture or dispersion with the bioresorbable polymer.

6. An implant material according to claim 4, wherein the bioceramic material is porous and comprises granules, flakes or powder.

7. An implant material according to claim 4, wherein the bioceramic material comprises pores of between about 10 and 1000 microns; preferably 15 and 500 microns; more preferably 20 and 300 microns.

8. An implant material according to claim 1, wherein the bioresorbable polymer granules include a core comprising a different material.

9. An implant material according to claim 8, wherein the core comprises a bioceramic material.

10. An implant material according to claim 8, wherein the core comprises an inner core and an outer core, wherein the inner core comprises a bioceramic material and the outer core comprises a second bioresorbable polymer.

11. An implant material according to claim 10, wherein the outer core comprises a bioactive agent, a therapeutic agent, or a combination thereof.

12. An implant material according to claim 1, wherein the implant material comprises a bioactive agent, a therapeutic agent, or a combination thereof.

13. An implant material according to claim 12, wherein the bioresorbable polymer granules comprise the bioactive agent, the therapeutic agent, or the combination thereof.

14. An implant material according to claim 12, wherein the bioactive or therapeutic agent comprises at least one agent selected from the group consisting of a growth factor including bone morphogenic protein (BMP), platelet derived growth factor (PDGF), growth hormone, transforming growth factor-beta (TGF-beta), and insulin-like growth factor, a bone anabolic agent including parathyroid hormone and teriparatide, an anti-resorptive agent including a bisphosphonate, including etidronate, clodronate, tiludronate, pamidronate, neridronate, olpadronate, alendronate, ibandronate, risedronate, and zoledronate, and strontium ranelate, an antibiotic including gentamicin, vancomycin, tobramycin, erythromycin, and clindamycin, an anti-cancer drug including paclitaxel, and mercatopurine, and an anti-inflammatory and/or analgesic agent including acetylsalicylic acid, ibuprofen, naproxen, indomethacine, ketoprofen and diclofenac.

15. An implant material according to claim 10, wherein the second bioresorbable polymer of the outer core is less soluble in the biocompatible solvent than the bioresorbable polymer granules.

16. An implant material according to claim 4, wherein the bioceramic material comprises at least one selected from the group consisting of calcium phosphate including hydroxyapatite, substituted hydroxyapatite including silicon, carbonate, magnesium, strontium, and fluoride, tricalcium phosphate, biphasic calcium phosphate, tetracalcium phosphate, octacalcium phosphate, dicalcium phosphate dihydrate (brushite), dicalcium phosphate (monetite), calcium pyrophosphate, calcium pyrophosphate dihydrate, heptacalcium phosphate, calcium phosphate monohydrate, calcium sulphate, bioactive glass, and glass ceramic.

17. An implant material according to claim 1, wherein the bioresorbable polymer granules comprise at least one selected from the group consisting of a polymer comprising a poly-alpha-hydroxyacid group including poly(lactic acid), poly(glycolic acid), poly-L-lactide, poly-DL-lactide, poly(lactide-co-glycolide), poly(lactide-co-caprolactone), poly(L-lactide-co-DL-lactide), polycaprolactone, and a bioresorbable polyanhydride including polyamide, polyorthoester, polydioxanone, polycarbonate, polyaminoacid, poly(amino-ester), poly(amido-carbonate), polyphosphazene, polyether, polyurethane, and polycyanoacrylate.

18. An implant material according to claim 10, wherein the second bioresorbable polymer of the outer core is at least one selected from the group consisting of a polymer comprising a poly-alpha-hydroxyacid group including poly(lactic acid), poly(glycolic acid), poly-L-lactide, poly-DL-lactide, poly(lactide-co-glycolide), poly(lactide-co-caprolactone), poly(L-lactide-co-DL-lactide), and polycaprolactone, a bioresorbable polyanhydride including polyamide, polyorthoester, polydioxanone, polycarbonate, polyaminoacid, poly(amino-ester), poly(amido-carbonate), polyphosphazene, polyether, polyurethane, and polycyanoacrylate, and a polysaccharide including alginate, chitosan, carboxymethyl cellulose, hydroxypropylmethyl cellulose, dextran, and hyaluronic acid.

19. An implant material according to claim 1, wherein the biocompatible, water miscible solvent comprises at least one selected from the group consisting of N-methyl-pyrollidone, dimethyl sulphoxide, acetone, poly(ethylene glycol), tetrahydrofuran, isopropanol, and caprolactone.

20. An implant material according to claim 1, wherein the implant material includes a water soluble porogen that is not soluble in the biocompatible solvent.

21. An implant material according to claim 20, wherein the water soluble porogen comprises at least one selected from the group consisting of a soluble inorganic salt including sodium chloride, calcium chloride, strontium chloride, and magnesium chloride, a soluble organic compound including sucrose, glucose, lactose, calcium gluconate, and calcium lactate. a water soluble polymer including poly(ethylene glycol), poly(ethylene oxide), poly(ethylene oxide-b-propylene oxide), poly(vinyl alcohol), poly vinyl acetate, polyacrylic acid, poly vinyl pyrrolidone, and poly(vinyl phosphonic acid), and polysaccharide including carboxymethylcellulose, sodium alginate, chitosan, and dextran.

22. An implant material according to claim 1, wherein the implant material has an open porosity of greater than 15%.

23. An implant material according to claim 22, wherein the implant material has an open porosity of between about 15%-70%; more preferably about 20%-55%; most preferably about 25%-45%.
